(19) United States
(12) Patent Application Publication
(10) Pub. No.: US 2006/0039890 A1
(43) Pub. Date: Feb. 23, 2006

(54) Treatment of psychological and cognitive disorders using a cholesterol -lowering agent in combination with an antidepressant

(76) Inventors: Perry F. Renshaw, Arlington, MA (US); Bruce M. Cohen, Lexington, MA (US)

Correspondence Address: CLARK & ELBING LLP, 101 FEDERAL STREET, BOSTON, MA, 02110, US

(21) Appl. No.: 10/922,652

(22) Filed: Aug. 20, 2004

(51) Int. Cl. A61K 31/785; A61K 31/5415; A61K 31/401; A61K 31/366; A61K 31/192

(52) U.S. Cl. 424/78.16; 514/225.8; 514/423; 514/460; 514/548; 514/571

(57) ABSTRACT

The present invention features compositions, kits, and methods for treating or reducing a cognitive or psychological disorder, such as depression.

### FIELD OF THE INVENTION

The invention relates to methods for treating psychological and cognitive disorders by administering a cholesterol-lowering agent in combination with an antidepressant.

### BACKGROUND

Cell membranes define the boundaries of cells and perform a variety of important cellular functions. One of their primary functions is to control the traffic of substances into and out of the cell. They also play a vital role in cell-cell recognition, adhesion, communication, and signaling.

The composition of a cell membrane determines its microscopic structure, which in turn, affects such parameters as membrane shape, permeability, and fluidity, as well as the conformation and functionality of ion channels, enzymes, and receptors that are embedded within the membrane. Lipids and proteins are the primary components of cell membranes, although carbohydrates may also be present. Phospholipids, such as phosphatidylcholine and sphingomyelin, are the most abundant membrane lipids. Glycolipids and cholesterol are also prevalent in cell membranes.

Alterations in membrane lipid order and composition can have a profound impact on the physical and chemical properties of the membrane. For instance, changes in the membrane's cholesterol-to-phospholipid ratio can lead to changes in membrane fluidity. Generally, an increase in cholesterol content results in a decrease in membrane fluidity, while a reduction in membrane cholesterol tends to increase fluidity. Even relatively small changes in membrane fluidity can induce considerable effects on membrane-linked functions, including ion transport, signal recognition and transduction, and the regulation of enzyme activities.

The relationship between cognitive and psychological disorders, such as depression, and neurobiology is extremely complex and ameliorating the cause or symptoms of a cognitive or psychological disorder appears to involve more than simply increasing the availability of neurotransmitters. For example, patients receiving antidepressants, which are drugs that alter the uptake of neurotransmitters, such as serotonin and dopamine, by neurons in the brain, demonstrate an immediate increase in the availability of various neurotransmitters, yet mood elevation can take months of pharmacotherapy, indicating an adaptation or drug-induced plasticity is taking place. For some patients, antidepressant therapy does not relieve the symptoms of their cognitive or psychological disorder. Accordingly, there is a need for improved therapies that provide relief to patients diagnosed with cognitive or psychological disorders.

### SUMMARY OF THE INVENTION

The method of the present invention involves the following steps: (a) performing a diagnostic test on the patient to determine that the patient has a cognitive or psychological disorder and, if the patient has been so diagnosed, (b) administering to the patient a cholesterol-lowering agent in combination with an antidepressant. The cholesterol-lowering agent is administered in an amount sufficient to lower the serum cholesterol of the patient and to increase brain membrane fluidity. The cholesterol-lowering agent and the antidepressant work synergistically to treat or reduce the severity of the cognitive or psychological disorder of the patient.

In an embodiment, the cholesterol-lowering agent and the antidepressant are administered in separate formulations. Alternatively, the cholesterol-lowering agent and the antidepressant can be admixed together in a single formulation. When administered in separate formulations, the agents may be administered simultaneously or within 28 days, 14 days, 7 days, or 1 day of each other. The cholesterol-lowering agent and the antidepressant may or may not be administered by the same route of administration (e.g., oral, intravenous, intramuscular, ophthalmic, topical, dermal, sub-cutaneous, and rectal). Optionally, the method can include an additional therapeutic regimen, such as a lifestyle change, including the adoption of a low-fat diet or low-sodium diet, stress management, physical exercise, reduction in alcohol intake, and reduction in smoking.

For example, the patient being treated may be administered the cholesterol-lowering agent and the antidepressant within 28 days of each other in amounts that together are sufficient to treat or reduce the cognitive or psychological disorder.

The cholesterol-lowering agent can be selected from the group consisting of a fibrate (e.g., clofibrate (ATROMID-S®)), a bile acid sequestrant (e.g., cholestyramine and colestipol (COLESTID®), and nicotinic acid (niacin)), gemfibrozil (LOPID® and GEMCOR®), probucol (PANAVIR®), and an HMG-CoA reductase inhibitor (e.g., a statin, such as lovastatin (MEVACOR®), cerivastatin (BAYCOL®), fluvastatin (LESCOL®), atorvastatin (LIPITOR®), pravastatin (PRAVACHOL®), and simvastatin (ZOCOR®)). Preferably, the agent is atorvastatin or simvastatin.

The antidepressant can be selected from the group consisting of a tricyclic antidepressant (TCA; e.g., imipramine (TOFRANIL® and others), amitriptyline (ELAVIL® and ENDEP®), amoxapine, desipramine (NORPRAMINE® and PERTOFRANE®), nortriptyline (PAMELOR® and AVENTYL®), trimipramine (SURMONTIL®), protriptyline (VIVACTIL®), doxepin (ADAPIN®, SINEQUAN®), clomipramine (ANAFRANIL®), and maprotiline), a selective serotonin reuptake inhibitor (SSRI; e.g., fluoxetine (PROZAC®), duloxetine (CYMBALTA®), sertraline (ZOLOFT®), paroxetine (PAXIL® and SEROXAT®), fluvoxamine (LUVOX®), citalopram (CELEXA®), escitalopram (LEXAPRO®), and cipralex (ESCITALOPRAM®), a serotonin and noradrenaline reuptake inhibitor (SNRI; e.g., milnacipran (IXEL®), venlafaxine (EFFEXOR®), trazodone (DESYREL®), mirtazapine (REMERON®), nefazodone (SERZONE®), reboxetine (EDRONAX® and VESTRA®), and bupropion (WELLBUTRIN®), and a monoamine oxidase inhibitor (MAOI; e.g., phenelzine (NARDIL®), tranylcypromine (PARNATE®), isocarboxazid (MARPLAN®), moclobemide (AUROIX® and MANERIX®), brofaromine (CONSONAR®), selegiline (ATAPRYL®, DEPRENYL®, and ELDEPRYL®), furazolidone (FUROXONE®), isoniazid (LANIAZID® and NYDRAZID®), isoniazid rifampin (RIFAMATE® and RIMACTANE®/INH), pargyline (EUTONYL®), procarbazine (MATULANE®), nomifensine (MERITAL®), FA70, clorgyline, TV3326 (N-propargyl-(3R)-aminoindan-5-yl-ethyl methylcarbamte hemitartate), and befloxatone).

The method of the invention may be used to treat or reduce the severity of a variety of cognitive and psychological disorders, including cognitive and affective disorders, such as depression (e.g., treatment-related depression), dysthymia, cyclothymia, bipolar disorder, schizophrenia and schizoaffective disorder, borderline personality disorder, age-related memory loss, mild cognitive impairment, and dementia of any etiology (e.g., Alzheimer Disease, Parkinson's Disease, Creutzfeldt-Jakob Disease, Huntington's Disease, Pick's Disease, HIV, head trauma), panic disorder, social phobia, bulimia, narcolepsy, attention deficit disorder (ADD; with or without hyperactivity), obsessive-compulsive disorder, and substance abuse disorders, including alcohol, stimulant, opiate, marijuana, solvent, and nicotine abuse or dependence. The disorder may develop independent of a particular treatment regimen or it may be caused by or related to treatment that a patient is receiving for a cognitive, psychological, or other disease or disorder.

In a further aspect, the present invention features a kit that includes a cholesterol-lowering agent selected from a fibrate (e.g., clofibrate (ATROMID-S®)), a bile acid sequestrant (e.g., cholestyramine and colestipol (COLESTID®), and nicotinic acid (niacin)), gemfibrozil (LOPID® and GEMCOR®), probucol (PANAVIR®), and an HMG-CoA reductase inhibitor (e.g., lovastatin (MEVACOR®), cerivastatin (BAYCOL®), fluvastatin (LESCOL®), atorvastatin (LIPITOR®), pravastatin (PRAVACHOL®), and simvastatin (ZOCOR®)) and instructions for its administration, separately or in admixture, with an antidepressant to a patient having or at risk of having a cognitive or psychological disorder.

The invention also features a kit that includes an antidepressant selected from a tricyclic antidepressant (TCA; e.g., imipramine (TOFRANIL® and others), amitriptyline (ELAVIL® and ENDEP®), amoxapine, desipramine (NORPRAMINE® and PERTOFRANE®), nortriptyline (PAMELOR® and AVENTYL®), trimipramine (SURMONTIL®), protriptyline (VIVACTIL®), doxepin (ADAPIN®, SINEQUAN®), clomipramine (ANAFRANIL®), and maprotiline), a selective serotonin reuptake inhibitor (SSRI; e.g., fluoxetine (PROZAC®), duloxetine (CYMBALTA®), sertraline (ZOLOFT®), paroxetine (PAXIL® and SEROXAT®), fluvoxamine (LUVOX®), citalopram (CELEXA®), escitalopram (LEXAPRO®), and cipralex (ESCITALOPRAM®), a serotonin and noradrenaline reuptake inhibitor (SNRI; e.g., milnacipran (IXEL®), venlafaxine (EFFEXOR®), trazodone (DESYREL®), mirtazapine (REMERON®), nefazodone (SERZONE®), reboxetine (EDRONAX® and VESTRA®), and bupropion (WELLBUTRIN®), and a monoamine oxidase inhibitor (MAOI; e.g., phenelzine (NARDIL®), tranylcypromine (PARNATE®), isocarboxazid (MARPLAN®), moclobemide (AUROIX® and MANERIX®), brofaromine (CONSONAR®), selegiline (ATAPRYL®, DEPRENYL®, and ELDEPRYL®), furazolidone (FUROXONE®), isoniazid (LANIAZID® and NYDRAZID®), isoniazid rifampin (RIFAMATE® and RIMACTANE®/INH), pargyline (EUTONYL®), procarbazine (MATULANE®), nomifensine (MERITAL®), FA70, clorgyline, TV3326 (N-propargyl-(3R)-aminoindan-5-yl-ethyl methylcarbamte hemitartate), and befloxatone) and instructions for its administration, separately or in admixture, with a cholesterol-lowering agent to a patient having or at risk of having a cognitive or psychological disorder.

The invention also features a kit that includes a cholesterol-lowering agent selected from a fibrate (e.g., clofibrate (ATROMID-S®)), a bile acid sequestrant (e.g., cholestyramine and colestipol (COLESTID®), and nicotinic acid (niacin)), gemfibrozil (LOPID® and GEMCOR®), probucol (PANAVIR®), and an HMG-CoA reductase inhibitor (e.g., lovastatin (MEVACOR®), cerivastatin (BAYCOL®), fluvastatin (LESCOL®), atorvastatin (LIPITOR®), pravastatin (PRAVACHOL®), and simvastatin (ZOCOR®)) and an antidepressant selected from a tricyclic antidepressant (TCA; e.g., imipramine (TOFRANIL® and others), amitriptyline (ELAVIL® and ENDEP®), amoxapine, desipramine (NORPRAMINE® and PERTOFRANE®), nortriptyline (PAMELOR® and AVENTYL®), trimipramine (SURMONTIL®), protriptyline (VIVACTIL®), doxepin (ADAPIN®, SINEQUAN®), clomipramine (ANAFRANIL®), and maprotiline), a selective serotonin reuptake inhibitor (SSRI; e.g., fluoxetine (PROZAC®), duloxetine (CYMBALTA®), sertraline (ZOLOFT®), paroxetine (PAXIL® and SEROXAT®), fluvoxamine (LUVOX®), citalopram (CELEXA®), escitalopram (LEXAPRO®), and cipralex (ESCITALOPRAM®), a serotonin and noradrenaline reuptake inhibitor (SNRI; e.g., milnacipran (IXEL®), venlafaxine (EFFEXOR®), trazodone (DESYREL®), mirtazapine (REMERON®), nefazodone (SERZONE®), reboxetine (EDRONAX® and VESTRA®), and bupropion (WELLBUTRIN®), and a monoamine oxidase inhibitor (MAOI; e.g., phenelzine (NARDIL®), tranylcypromine (PARNATE®), isocarboxazid (MARPLAN®), moclobemide (AUROIX® and MANERIX®), brofaromine (CONSONAR®), selegiline (ATAPRYL®, DEPRENYL®, and ELDEPRYL®), furazolidone (FUROXONE®), isoniazid (LANIAZID® and NYDRAZID®), isoniazid rifampin (RIFAMATE® and RIMACTANE®/INH), pargyline (EUTONYL®), procarbazine (MATULANE®), nomifensine (MERITAL®), FA70, clorgyline, TV3326 (N-propargyl-(3R)-aminoindan-5-yl-ethyl methylcarbamte hemitartate), and befloxatone) and instructions for their administration, separately or in admixture, to a patient having or at risk of having a cognitive or psychological disorder.

Other features and advantages of the invention will be apparent from the following detailed description thereof and from the claims.

### Definitions

By “affective disorder” is meant any emotional or mental disorder characterized primarily by disturbances in mood.

By “an amount sufficient,” when referring to a cholesterol-lowering agent, is meant the amount of a chemical compound or composition, alone or in combination with another therapeutic regimen, is capable of lowering the serum cholesterol level of a human by at least 5%, 10%, 15%, 20%, 25%, 30%, 35%, 40%, 45%, 50%, or more as measured by any standard technique for determining cholesterol level (e.g., LDL cholesterol, HDL cholesterol, and triglycerides). A sufficient amount of a cholesterol-lowering agent used to practice the present invention for therapeutic treatment of cognitive or psychological disorders varies depending upon the manner of administration, the age, body weight, and general health of the patient. Ultimately, the prescribers will decide the appropriate amount and dosage regimen.

When referring to an antidepressant, “an amount sufficient” means that a chemical compound or composition, alone or in combination with another therapeutic regimen, is capable of effecting treatment or a reduction in the severity of, e.g., depression.

By “antidepressant” is meant a chemical compound or composition capable of modulating the action of a neurotransmitter (e.g., norepinephrine, serotonin, and dopamine) at the synapse of neurons (i.e., by increasing the release of a neurotransmitter, decreasing the uptake or degradation of a neurotransmitter, activating a neurotransmitter signaling receptor, or down-regulating a neurotransmitter reuptake receptor).

By “cholesterol-lowering agent” is meant a chemical compound or composition capable of lowering the serum cholesterol level of a patient (e.g., a human or non-human animal) by at least 5%, 10%, 15%, 20%, 25%, 30%, 35%, 40%, 45%, 50%, or more as measured by any standard technique for determining cholesterol level (e.g., LDL cholesterol, HDL cholesterol, and triglycerides) relative to a patient not administered the chemical compound or composition.

By “cognitive disorder” is meant any disorder that affects mental processes, including impairments of memory, learning, awareness, attention, communication, intellectual capacity, judgment-making ability, and/or motor coordination. Such disorders are often accompanied by personality and behavioral changes. Examples of these disorders include, but are not limited to delirium, dementia, and amnestic disorders.

As used herein, the meaning of the term “depression” is consistent with its accepted meaning in the art (see, e.g., DSM-IV[R] and The Merck Manual, Beers, M. H., et al., eds., 1531-1538 (17th ed., 1999). Psychological symptoms of depression include, but are not limited to, changes in mood, feelings of intense sadness, despair, mental slowing, loss of concentration, pessimistic worry, agitation, and self-deprecation. Physical symptoms of depression include, but are not limited to, insomnia, anorexia, weight loss, decreased energy, and abnormal hormonal circadian rhythms. As used herein, the terms “treatment or reduction in the severity of depression” and “treating depression” mean the relief from, or reduction in the psychological or physical symptoms of, depression.

By “membrane fluidity-related” is meant associated with a change (either a decrease or increase) in cell membrane fluidity or membrane order.

By “substance abuse or addiction” is meant the physical and/or psychological addiction to, or dependence on, a substance. Examples of substances to which a patient can be addicted or dependent include, but are not limited to: central nervous system (CNS) depressants, such as alcohol, barbiturates, ethchlorvynol, glutethimide, methaqualone, methyprylon, and natural and synthetic opiates; anxiolytics, such as alprazolam, oxazepam, temazepam, chlordiazepoxide, and diazepam; stimulants, such as amphetamines and methamphetamine, in particular, nicotine, and cocaine; and hallucinogens, such as LSD, marijuana, and mescaline. Psychological symptoms of substance addiction include, but are not limited to, feelings of satisfaction and a desire to repeat the drug experience, craving of the substance, and compulsive use of the substance. Psychological symptoms of substance (i.e., drug or alcohol) withdrawal include, but are not limited to, hallucinations and symptoms of depression and anxiety. Physical symptoms of substance addiction include, but are not limited to, the physical symptoms of depression discussed above. Physical symptoms of drug withdrawal include pain and the physical symptoms of depression discussed above.

By “substance abuse disorder” is meant any physiological or psychological disorder characterized primarily by the abuse of, addiction to, or dependence on a chemical substance.

By “treating or reducing the severity of a cognitive or psychological disorder” is meant ameliorating such a condition has been diagnosed. As compared with an equivalent untreated control, such treatment or reduction of severity is at least 5%, 10%, 20%, 40%, 50%, 60%, 80%, 90%, 95%, or 100% as measured by any standard technique. A patient who is being treated for a cognitive or psychological disorder is one who a medical practitioner has diagnosed as having such a condition. Diagnosis may be performed by any suitable means, such as those described herein. One in the art will understand that patients of the invention may have been subjected to standard tests or may have been identified, without examination, as one at high risk due to the presence of one or more risk factors, such as family history, high blood levels of triglycerides, high blood levels of cholesterol, and the presence of molecular markers.

### DETAILED DESCRIPTION

The present invention provides a method of treating cognitive and/or psychological disorders that are associated with a decrease in brain cell membrane fluidity. The method involves administering to a patient a cholesterol-lowering agent in combination with an antidepressant. The cholesterol-lowering agent reduces the patient's level of serum cholesterol, thereby producing a corresponding decrease in the level of cholesterol in neuronal cell membranes and a concomitant increase in membrane fluidity. The increase in membrane fluidity significantly enhances the therapeutic effect of the co-administered antidepressant. Thus, the cholesterol-lowering agent and the antidepressant, when administered to a patient diagnosed with a cognitive or psychological disorder, produces a synergistic effect that eliminates or reduces the severity of the adverse effects associated with the patient's cognitive or psychological disorder to a greater extent than the effect either agent alone would have on a patient diagnosed with a cognitive or psychological disorder.

Diagnosis of Psychological and Cognitive Disorders

The initial step of this method involves diagnosing a human patient to determine whether the individual is suffering from a condition that is associated with a cognitive or psychological disorder that is linked to membrane fluidity abnormalities. A number of psychological and cognitive disorders are marked by changes in the lipid composition of brain cell membranes, which result in a decrease in membrane fluidity. This reduction in membrane fluidity can lead to a variety of mental impairments due to the improper transport of proteins and/or signaling events. The administration of cholesterol-lowering agents restores proper membrane fluidity and, consequently, improves diffusion and transport of biologically-active molecules and receptors through the cell membrane, thereby facilitating cell signaling and amplifying the action of co-administered agents, such as antidepressants. Examples of psychological disorders, which may be characterized by a decrease in membrane fluidity, include, but are not limited to, affective disorders, such as major depression, treatment-related depression, dysthymia, cyclothymia, bipolar disorder, schizoprenia and schizoaffective disorder, borderline personality disorder, panic disorders, social phobias, bulimia, narcolepsy, and obsessive-compulsive disorder.

Cognitive disorders are often age-related or the result of neurodegenerative disease processes, and can also be accompanied by changes in neuronal membrane lipid composition and fluidity. Some of the adverse symptoms of these disorders may stem from a decrease in brain cell membrane fluidity, and can therefore be treated by administration of cholesterol-lowering agents. Exemplary membrane fluidity-related cognitive disorders include, but are not limited to, age-related memory loss, mild cognitive impairment, dementia of any etiology (e.g., dementia caused by or associated with Alzheimer Disease, Parkinson's Disease, Creutzfeldt-Jakob Disease, Huntington's Disease, Pick's Disease, human immunodeficiency virus (HIV) infection, autoimmune deficiency syndrome (AIDS), and head trauma), and attention deficit disorder (with or without hyperactivity).

Overuse of certain psychoactive substances may also effect brain cell membrane composition and fluidity. The method of the invention may, therefore, be useful in the treatment of various substance abuse disorders in which the patient abuses or is dependent on a substance that includes, but is not limited to, a depressant, an anxiolytic, a stimulant, a hallucinogen, and a solvent. Examples of depressants include, e.g., alcohol, barbiturates, ethchlorvynol, glutethimide, methaqualone, methyprylon, and opiates. Examples of anxiolytics include, e.g., alprazolam, oxazepam, temazepam, chlordiaazepoxide, and diazepam. Examples of stimulants include, e.g., amphetamines, methamphetamines, cocaine, and nicotine. Examples of hallucinogens include, e.g., lysergic acid diethylamide (LSD), marijuana, and mescaline.

Cognitive and psychological disorders, including affective disorders and substance abuse disorders, can be diagnosed using a variety of well-known testing procedures. Two commonly used systems for diagnosing such disorders are the Diagnostic and Statistical Manual of Mental Disorders (DSM-IV) and the International Classification of Disease (ICD-10). These systems provide a set of standard criteria for effectively and reliably diagnosing a broad range of mental disorders. In some circumstances, biochemical and serological methods may also be available for diagnosing these disorders, and may be used alone or in conjunction with other diagnostic methods, including psychological testing. Of course, the method of diagnosis will vary depending on the condition of the patient and the nature of the disorder being diagnosed.

Therapeutic Administration

Once a patient has been diagnosed with a cognitive or psychological disorder, the patient is treated by administration of a cholesterol-lowering agent and an antidepressant. The cholesterol-lowering agent restores proper brain cell membrane fluidity, thereby alleviating the symptoms associated with decreased fluidity and enhancing the therapeutic action of the co-administered antidepressant.

A wide variety of cholesterol-lowering agents and antidepressants are known in the art and may be used in the present invention. Examples of suitable cholesterol-lowering agents include, but are not limited to, fibrates, which are hypolipidemic agents that activate the peroxisome proliferator-activated receptor α (PPARα) and regulate the expression of genes involved in lipid metabolism (e.g., clofibrate (ATROMID-S®)), bile acid sequestrants (e.g., cholestyramine and colestipol (COLESTID®), and nicotinic acid (niacin)), gemfibrozil (LOPID® and GEMCOR®), probucol (PANAVIR®), and HMG-CoA reductase inhibitors, e.g., statins, such as fluvastatin (LESCOL®), atorvastatin (LIPITOR®), pravastatin (PRAVACHOL®), lovastatin (MEVACOR®), cerivastatin (BAYCOL®), and simvastatin (ZOCOR®). Methods for preparing these and other cholesterol-lowering agents are well known in the art and many are commercially available medications.

Examples of suitable antidepressants include, but are not limited to, tricyclic antidepressants (TCAs; e.g., imipramine (TOFRANIL® and others), amitriptyline (ELAVIL® and ENDEP®), amoxapine, desipramine (NORPRAMINE® and PERTOFRANE®), nortriptyline (PAMELOR® and AVENTYL®), trimipramine (SURMONTIL®), protriptyline (VIVACTIL®), doxepin (ADAPIN®, SINEQUAN®), clomipramine (ANAFRANIL®), and maprotiline), selective serotonin reuptake inhibitors (SSRIs; e.g., fluoxetine (PROZAC®), duloxetine (CYMBALTA®), sertraline (ZOLOFT®), paroxetine (PAXIL® and SEROXAT®), fluvoxamine (LUVOX®), citalopram (CELEXA®), escitalopram (LEXAPRO®), and cipralex (ESCITALOPRAM®), serotonin and noradrenaline reuptake inhibitors (SNRIs; e.g., milnacipran (IXEL®), venlafaxine (EFFEXOR®), trazodone (DESYREL®), mirtazapine (REMERON®), nefazodone (SERZONE®), reboxetine (EDRONAX® and VESTRA®), and bupropion (WELLBUTRIN®), and monoamine oxidase inhibitors (MAOIs; e.g., phenelzine (NARDIL®), tranylcypromine (PARNATE®), isocarboxazid (MARPLAN®), moclobemide (AUROIX® and MANERIX®), brofaromine (CONSONAR®), selegiline (ATAPRYL®, DEPRENYL®, and ELDEPRYL®), furazolidone (FUROXONE®), isoniazid (LANIAZID® and NYDRAZID®), isoniazid rifampin (RIFAMATE® and RIMACTANE®/INH), pargyline (EUTONYL®), procarbazine (MATULANE®), nomifensine (MERITAL®), FA70, clorgyline, TV3326 (N-propargyl-(3R)-aminoindan-5-yl-ethyl methylcarbamte hemitartate), and befloxatone).

The cholesterol-lowering agent and antidepressant may be delivered separately or may be admixed into a single formulation. When the cholesterol-lowering agent and antidepressant are present in different pharmaceutical compositions, different routes of administration may be employed. Routes of administration for the various embodiments include, but are not limited to, topical, transdermal, and systemic administration (such as, intravenous, intraarterial, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. Desirably, the cholesterol-lowering agent and antidepressant of the invention are administered within at least 1, 2, 4, 6, 10, 12, 18, 24 hours, 3 days, 7 days, 14 days, or 28 days apart. The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

The cholesterol-lowering agent and antidepressant can be administered in admixture with a pharmaceutically acceptable carrier adapted for the route of administration. A variety of physiologically acceptable carriers can be used to administer the cholesterol-lowering agent and antidepressant and their formulations are known to those skilled in the art and are described in, for example, Remington: The Science and Practice of Pharmacy, 20th edition, 2000, ed. A. R. Gennaro, Lippincott Williams & Wilkins, Philadelphia, Encyclopedia of Pharmaceutical Technology, eds. J. Swarbrick and J. C. Boylan, 1988-1999, Marcel Dekker, New York, and the Merck Index, Merck & Co., Rahway, N.J.

Oral ingestion is the preferred route of administration. Compositions intended for oral use can be prepared in solid or liquid forms, according to any method known to the art for the manufacture of pharmaceutical compositions. The compositions may optionally contain sweetening, flavoring, coloring, perfuming, and preserving agents in order to provide a more palatable preparation. Solid dosage forms for oral administration include capsules, tablets, pills, powders, and granules. Generally, these pharmaceutical preparations contain active ingredients admixed with non-toxic pharmaceutically acceptable excipients. These may include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium, and can also include adjuvants, such as wetting agents, emulsifying agents, and suspending agents.

Alternatively, the pharmaceutical compositions can be administered parenterally (e.g., by intramuscular, intraperitoneal, intravenous, or subcutaneous injection or implant). Formulations for parenteral administration include sterile aqueous or non-aqueous solutions, suspensions, or emulsions. A variety of aqueous carriers can be used, e.g., water, buffered water, 0.4 percent saline, and the like. Examples of other suitable vehicles include polypropylene glycol, polyethylene glycol, vegetable oils, gelatin, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

The cholesterol-lowering agent and antidepressant can also be administered in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

The cholesterol-lowering agent and antidepressant may be formulated in a variety of ways that are known in the art. Desirably, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Dosage

The amount of active ingredient that is combined with the carrier materials to produce a single dosage will vary depending upon the subject being treated and the particular mode of administration. Generally, the cholesterol-lowering agent should be administered in an amount sufficient to lower the serum cholesterol level of the patient. The level should be lowered sufficiently to increase the patient's brain cell membrane fluidity. Membrane fluidity can be monitored using T2 MR mapping, a technique that indirectly measures the physical properties of the outer leaflet of the lipid bilayer of cell membranes and is described in U.S. Ser. No. 60/254,279, which is hereby incorporated by reference. T2 mapping works by measuring the relative movement of water molecules in the immediate vicinity of the cell membrane. A decrease in the amount of cholesterol incorporated into the cell membrane, which would result in an increase in fluidity, would be observed as a change in the T2 signal.

Generally, the antidepressant should be administered in an amount sufficient to modulate the action of a neurotransmitter (i.e., by increasing the release of neurotransmitters, decreasing the uptake or degradation of neurotransmitters, activating neurotransmitter signaling receptors, or down-regulating neurotransmitter reuptake receptors). Methods for determining the amount of antidepressant sufficient to modulate the action of a neurotransmitter can be found in, e.g., U.S. Pat. No. 6,700,018, incorporated herein by reference. The amount of cholesterol-lowering agent and antidepressant administered should be sufficient to cure or at least partially arrest the symptoms of the cognitive or psychological disorder and its complications.

Dosage levels on the order of about 0.1 mg to about 400 mg per kilogram of body weight per day (from about 1.0 mg to about 30.0 g per 70 kg patient per day) are useful in the treatment of the above mentioned psychological and cognitive disorders. The daily dosage may be administered as a single dose or divided into multiple doses. Typically, patients take one or two capsules orally, three to four times per day (e.g., once in the morning, once in the early afternoon, and again in the evening). In general, the desired daily dosage should be taken for a prolonged period, usually at least two weeks, preferably four to six weeks, although longer periods of administration of two months or more may be needed.

Suitable dosage ranges for several well-known cholesterol-lowering agents are provided in the following table.

TABLE 1
Dosage ranges for commonly used cholesterol-lowering agents.

| | Daily Dosage Range | |
|---|---|---|
| Cholesterol-Lowering Agent | (per 70 kg of body weight) | |
| Clofibrate | 500 to 2000 | mg |
| Colestipol | 5 to 30 | g |
| Gemfibrozil | 1200 | mg |
| Fluvastatin | 20 to 40 | mg |
| Atorvastatin | 20 to 80 | mg |
| Simvastatin | 5 to 80 | mg |
| Pravastatin | 10 to 20 | mg |
| Lovastatin | 20 to 80 | mg |
| Cerivastatin | 0.2 to 0.8 | mg |

Suitable dosage ranges for several well-known antidepressant agents are provided in the following table.

TABLE 2
Dosage ranges for commonly used antidepressant agents.

| | Daily Dosage Range | |
|---|---|---|
| Antidepressant Agent | (per 70 kg of body weight) | |
| Imipramine | 100 to 250 | mg |
| Fluoxetine | 10 to 80 | mg |
| Sertraline | 20-70 | mg |
| Paroxetine | 10 to 40 | mg |
| Citalopram | 20 to 60 | mg |
| Venlafaxine | 75 to 375 | mg |
| Buproprion | 75 to 450 | mg |
| Phenelzine | 45 to 90 | mg |
| Trancylcypromine | 10 to 60 | mg |

One skilled in the art will appreciate that the exact individual dosages may be adjusted somewhat depending on a variety of factors, including the specific cholesterol-lowering agent and antidepressant being administered, the time of administration, the route of administration, the nature of the formulation, the rate of excretion, the particular disorder being treated, the severity of the disorder, and the age, weight, health, and gender of the patient. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well-known in the art. The precise therapeutically effective dosage levels and patterns are preferably determined by the attending physician in consideration of the above identified factors.

With respect to substance abuse disorders, the dose level for suppressing an urge to consume the abused substance may vary among individuals depending upon the severity of the individual's symptoms and/or the individual's predisposition or susceptibility to substance abuse. The optimum dosage can generally be determined by monitoring the amount of substance used by the individual while on the medication or by the intensity of the individual's desire for the abused substance.

In addition to treating pre-existing cognitive, affective, and substance abuse disorders, a cholesterol-lowering agent and an antidepressant can be administered in combination (formulated separately or in admixture) prophylactically in order to prevent or slow the onset of these disorders. In prophylactic applications, the cholesterol-lowering agent and antidepressant are administered to a patient susceptible to or otherwise at risk of a cognitive or psychological disorder that is caused by, or occurs as a result of, a decrease in membrane fluidity due to an increase in cholesterol levels in the cell membrane. Again, the precise amounts that are administered depend on various factors such as the patient's state of health and weight, but generally range from about 0.5 mg to about 5,000 mg per 70 kilogram patient, more commonly from about 5 mg to about 2,000 mg per 70 kg of body weight, and most commonly from about 10 mg to about 1000 mg per 70 kg of body weight.

Administration of each chemical compound or composition in the combination can, independently, be one to four times daily for one day to one year, and may even be for the life of the patient. Chronic, long-term administration will be indicated in many cases.

Determination of Treatment Efficacy or Reduction in the Severity of a Cognitive or Psychological Disorder

Several standardized rating scales are known in the art and can be used to determine whether a treatment regimen has treated or reduced the severity of the cognitive or psychological disorders disclosed herein.

For example, the determination of whether a treatment regimen has been effective for the treatment or reduction in the severity of depression can be made using, e.g., the Hamilton Depression Rating Scale, the Montgomery-Asburg Depression Rating Scale, or the Beck Depression Inventory.

A determination of whether cognition has been improved can be made using a cognitive testing battery. For example, the Mini Mental Status Examination (MMSE) can be used to screen for dementia or monitor its progression. The Alzheimer's Disease Assessment Scale—cognitive subscale (ADAS-Cog) can also be employed for testing an improvement in cognition and memory.

Other assessment tools that are designed to evaluate several areas of function, including, e.g., cognition, functional capacity, behavior, general physical health, and quality of life are indicated below.

Cognitive assessments: Blessed Information-Memory-Concentration Test (BIMC) and Clinical Dementia Rating Scale (CDR).

Functional assessments: Functional Assessment Questionnaire (FAQ), Instrumental Activities of Daily Living (IADL), Physical Self-Maintenance Scale (PSMS), and Progressive Deterioration Scale (PDS).

Global assessments: Clinical Global Impression of Change (CGIC), Clinical Interview-Based Impression (CIBI), and Global Deterioration Scale (GDS).

Caregiver-based assessments: Behavioral Pathology in Alzheimer's Disease Rating Scale (BEHAVE-AD), and Neuropsychiatric Inventory (NPI).

The appropriate test(s) can be selected and employed by the skilled artisan, who can evaluate the results of the test(s) to determine whether a treatment regimen has treated or reduced the severity of a patient's cognitive or psychological disorder.

### Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein.

All publications and patents mentioned in this specification are hereby incorporated by reference.

What is claimed is:

1. A composition comprising a cholesterol-lowering agent and an antidepressant, wherein said cholesterol-lowering agent and antidepressant are present in amounts that, when administered to a human, are sufficient to treat or reduce the severity of a cognitive or psychological disorder.

2. The composition of claim 1, wherein said cholesterol-lowering agent is selected from a fibrate, a bile acid sequestrant, nicotinic acid, gemfibrozil, probucol, and an HMG-CoA reductase inhibitor.

3. The composition of claim 2, wherein said fibrate is clofibrate.

4. The composition of claim 2, wherein said bile acid sequestrant is cholestyramine or colestipol.

5. The composition of claim 2, wherein said HMG-CoA reductase inhibitor is a statin.

6. The composition of claim 5, wherein said statin is lovastatin, cerivastatin, fluvastatin, atorvastatin, pravastatin, or simvastatin.

7. The composition of claim 1, wherein said antidepressant is selected from a tricyclic antidepressant, a selective serotonin reuptake inhibitor (SSRI), a serotonin and noradrenaline reuptake inhibitor (SNRI), and a monoamine oxidase inhibitor (MAOI).

8. The composition of claim 7, wherein said tricyclic antidepressant is imipramine, amitriptyline, amoxapine, desipramine, nortriptyline, trimipramine, protriptyline, doxepin, clomipramine, or maprotiline.

9. The composition of claim 7, wherein said SSRI is fluoxetine, duloxetine, sertraline, paroxetine, fluvoxamine, citalopram, escitalopram, or cipralex.

10. The composition of claim 7, wherein said SNRI is milnacipran, venlafaxine, trazodone, mirtazapine, nefazodone, reboxetine, or bupropion.

11. The composition of claim 7, wherein said MAOI is phenelzine, tranylcypromine, isocarboxazid, moclobemide, brofaromine, selegiline, furazolidone, isoniazid, isoniazid rifampin, pargyline, procarbazine, nomifensine, FA70, clorgyline, TV3326 (N-propargyl-(3R)-aminoindan-5-yl-ethyl methylcarbamte hemitartate), or befloxatone.

12. The composition of claim 1, wherein said cognitive or psychological disorder is selected from a cognitive disorder, an affective disorder, a neurobiological disorder, and a substance abuse disorder.

13. The composition of claim 12, wherein said cognitive disorder is selected from age-related memory loss, mild cognitive impairment, or dementia.

14. The composition of claim 13, wherein said dementia is caused by or associated with Alzheimer Disease, Parkinson's Disease, Creutzfeldt-Jakob Disease, Huntington's Disease, Pick's Disease, human immunodeficiency virus (HIV) infection, autoimmune deficiency syndrome (AIDS), or head trauma.

15. The composition of claim 12, wherein said affective disorder is depression, dysthymia, cyclothymia, bipolar disorder, schizophrenia, schizoaffective disorder, borderline personality disorder, panic disorder, a social phobia, bulimia, narcolepsy, or obsessive-compulsive disorder.

16. The composition of claim 15, wherein said depression is treatment-related depression.

17. The composition of claim 12, wherein said neurobiological disorder is attention deficit disorder.

18. The composition of claim 12, wherein said substance abuse disorder is characterized by an abuse of or dependence on a substance selected from a depressant, an anxiolytic, a stimulant, a hallucinogen, and a solvent.

19. The composition of claim 18, wherein said depressant is alcohol, a barbiturate, ethchlorvynol, glutethimide, methaqualone, methyprylon, or an opiate.

20. The composition of claim 18, wherein said anxiolytic is alprazolam, oxazepam, temazepam, chlordiaazepoxide, or diazepam.

21. The composition of claim 18, wherein said stimulant is an amphetamine, a methamphetamine, cocaine, or nicotine.

22. The composition of claim 18, wherein said hallucinogen is lysergic acid diethylamide (LSD), marijuana, or mescaline.

23. The composition of claim 1, further comprising a pharmaceutically acceptable carrier.

24. The composition of claim 1, wherein said composition is formulated for oral administration.

25. The composition of claim 1, wherein cholesterol-lowering agent and said antidepressant are provided in a solid formulation.

26. The composition of claim 25, wherein said solid formulation is a capsule, a tablet, a pill, a powder, or a granule.

27. The composition of claim 1, wherein cholesterol-lowering agent and said antidepressant are provided in a liquid formulation.

28. The composition of claim 27, wherein said liquid formulation is an emulsion, a solution, a suspension, a syrup, or a soft gelatin capsule.

29. The composition of claim 1, wherein said composition is formulated for systemic administration.

30. A method comprising:
(a) performing a diagnostic test on a patient to determine whether said patient has a cognitive or psychological disorder, and
b) if said patient is diagnosed with a cognitive or psychological disorder, administering to said patient a cholesterol-lowering agent and an antidepressant in amounts sufficient to treat or reduce the severity of said cognitive or psychological disorder.

31. The method of claim 30, wherein said cholesterol-lowering agent is selected from a fibrate, a bile acid sequestrant, nicotinic acid, gemfibrozil, probucol, and an HMG-CoA reductase inhibitor.

32. The method of claim 31, wherein said fibrate is clofibrate.

33. The method of claim 31, wherein said bile acid sequestrant is cholestyramine or colestipol.

34. The method of claim 31, wherein said HMG-CoA reductase inhibitor is a statin.

35. The method of claim 34, wherein said statin is lovastatin, cerivastatin, fluvastatin, atorvastatin, pravastatin, or simvastatin.

36. The method of claim 30, wherein said antidepressant is selected from a tricyclic antidepressant, a selective serotonin reuptake inhibitor (SSRI), a serotonin and noradrenaline reuptake inhibitor (SNRI), and a monoamine oxidase inhibitor (MAOI).

37. The method of claim 36, wherein said tricyclic antidepressant is imipramine, amitriptyline, amoxapine, desipramine, nortriptyline, trimipramine, protriptyline, doxepin, clomipramine, or maprotiline.

38. The method of claim 36, wherein said SSRI is fluoxetine, duloxetine, sertraline, paroxetine, fluvoxamine, citalopram, escitalopram, or cipralex.

39. The method of claim 36, wherein said SNRI is milnacipran, venlafaxine, trazodone, mirtazapine, nefazodone, reboxetine, or bupropion.

40. The method of claim 36, wherein said MAOI is phenelzine, tranylcypromine, isocarboxazid, moclobemide, brofaromine, selegiline, furazolidone, isoniazid, isoniazid rifampin, pargyline, procarbazine, nomifensine, FA70, clorgyline, TV3326 (N-propargyl-(3R)-aminoindan-5-yl-ethyl methylcarbamte hemitartate), or befloxatone.

41. The method of claim 30, wherein said cognitive or psychological disorder is selected from a cognitive disorder, an affective disorder, a neurobiological disorder, and a substance abuse disorder.

42. The method of claim 41, wherein said cognitive disorder is selected from age-related memory loss, mild cognitive impairment, or dementia.

43. The method of claim 42, wherein said dementia is caused by or associated with Alzheimer Disease, Parkinson's Disease, Creutzfeldt-Jakob Disease, Huntington's Disease, Pick's Disease, human immunodeficiency virus (HIV) infection, autoimmune deficiency syndrome (AIDS), or head trauma.

44. The method of claim 41, wherein said affective disorder is depression, dysthymia, cyclothymia, bipolar disorder, schizophrenia, schizoaffective disorder, borderline personality disorder, panic disorder, a social phobia, bulimia, narcolepsy, or obsessive-compulsive disorder.

45. The method of claim 44, wherein said depression is treatment-related depression.

46. The method of claim 41, wherein said neurobiological disorder is attention deficit disorder.

47. The method of claim 41, wherein said substance abuse disorder is characterized by an abuse of or dependence on a substance selected from a depressant, an anxiolytic, a stimulant, a hallucinogen, and a solvent.

48. The method of claim 47, wherein said depressant is alcohol, a barbiturate, ethchlorvynol, glutethimide, methaqualone, methyprylon, or an opiate.

49. The method of claim 47, wherein said anxiolytic is alprazolam, oxazepam, temazepam, chlordiaazepoxide, or diazepam.

50. The method of claim 47, wherein said stimulant is an amphetamine, a methamphetamine, cocaine, or nicotine.

51. The method of claim 47, wherein said hallucinogen is lysergic acid diethylamide (LSD), marijuana, or mescaline alcohol, a stimulant, an opiate, marijuana, a solvent, and nicotine.

52. The method of claim 30, wherein said cholesterol-lowering agent is administered in an amount sufficient to lower the serum cholesterol of, and increase membrane fluidity of neuronal cells in, said patient.

53. The method of claim 30, wherein said cholesterol-lowering agent or said antidepressant is formulated for oral administration.

54. The method of claim 30, wherein said cholesterol-lowering agent or said antidepressant is formulated for systemic administration.

55. The method of claim 30, wherein said cholesterol-lowering agent and said antidepressant are formulated as a single composition.

56. The method of claim 30, wherein said cholesterol-lowering agent and said antidepressant are formulated in two separate compositions.

57. The method of claim 56, wherein said cholesterol-lowering agent and said antidepressant are administered simultaneously, within 24 hours, or within 7, 14, or 28 days of each other.

58. The method of claim 57, wherein said cholesterol-lowering agent and said antidepressant are administered simultaneously.

59. The method of claim 57, wherein said cholesterol-lowering agent and said antidepressant are administered within 24 hours of each other.

60. The method of claim 57, wherein said cholesterol-lowering agent and said antidepressant are administered within 7 days of each other.

61. The method of claim 57, wherein said cholesterol-lowering agent and said antidepressant are administered within 14 days of each other.

62. The method of claim 57, wherein said cholesterol-lowering agent and said antidepressant are administered within 28 days of each other.

63. A kit comprising:
(a) a cholesterol-lowering agent in an amount sufficient to lower the serum cholesterol of, and increase the membrane fluidity of neuronal cells in, a patient administered said cholesterol-lowering agent; and
(b) instructions for administering said cholesterol-lowering agent and an antidepressant to a patient for the treatment of, or reduction in severity of, a cognitive or psychological disorder.

64. A kit comprising:
(a) an antidepressant; and
(b) instructions for administering said antidepressant and a cholesterol-lowering agent to a patient for the treatment of, or reduction in severity of, a cognitive or psychological disorder.

65. A kit comprising:
(a) a composition comprising a cholesterol-lowering agent and an antidepressant; and
(b) instructions for administering said composition to a patient for the treatment of, or reduction in severity of, a cognitive or psychological disorder.

66. A kit comprising:
(a) a cholesterol-lowering agent in an amount sufficient to lower the serum cholesterol of, and increase the membrane fluidity of neuronal cells in, a patient administered said cholesterol-lowering agent;
(b) an antidepressant; and
(c) instructions for administering said cholesterol-lowering agent and said antidepressant to a patient for the treatment of, or reduction in severity of, a cognitive or psychological disorder.
